(19) United States
(12) Patent Application Publication
(10) Pub. No.: US 2015/0362590 A1
(43) Pub. Date: Dec. 17, 2015

(54) CALCULATING VELOCITY OF MOVING OBJECTS WITH TIME OF FLIGHT OF ULTRASOUND PULSES AND RECTIFYING DETECTING DEPTH WITH REDUCED ULTRASOUND SPEED

(71) Applicant: Hai Huang, Herndon, VA (US)

(72) Inventor: Hai Huang, Herndon, VA (US)

(73) Assignee: Hai Huang, Herndon, VA (US)

(21) Appl. No.: 14/305,074

(22) Filed: Jun. 16, 2014

(51) Int. Cl. G01S 7/52; A61B 8/08; A61B 8/06

(52) U.S. Cl. CPC G01S 7/52017; A61B 8/06; A61B 8/488; A61B 8/5223

(57) ABSTRACT

During transmission the speed of ultrasound pulses gradually reduces due to their energy loss. So, calculating the detecting depth with fixed transmitting speed may distort two dimensional images due to the reduction of pulse speed. Correcting TOF error will rectify the depth registration and improve the quality of images.

The thickness of activated piezoelectric materials decides the size and quantity of ultrasound pulses, which is related to its penetrating ability. So, increasing thickness of activated piezoelectric materials will increase the quantity of ultrasound pulses, which increase their penetrating depth.

Moving objects change the speed of reflected ultrasound pulses. The forward moving objects increase reflected speed and reversely moving objects decrease reflected speed, which change their TOF. Therefore TOF shift can be used to calculate the velocity of moving objects in the continuous and pulse wave and color ultrasound, and correct aliasing of pulse and color ultrasound.

### CROSS REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

The transmission of ultrasound pulses is actually the energy traveling of acoustic pulses in the medium. Currently, it is supposed that the speed of ultrasound pulses is fixed in the same medium during transmission. Actually, the energy of ultrasound pulses is gradually reduced due to the acoustic impedance of transmitting medium. The acoustic impedance is directly related to the speed of ultrasound pulses in the medium. So, the question is if the speed of ultrasound pulses can still keep the same when its energy is gradually lower until exhausted?

Ultrasound pulses can be reflected by motionless or moving objects, and it is considered that forward moving objects can compress ultrasound frequency and reversely moving objects decompress the ultrasound frequency. So, Doppler has been widely used to measure the velocity of moving objects based on frequency shift, such as medical ultrasound machine and Doppler radar. In pulse wave Doppler, aliasing is explained with insufficient Doppler sampling rate of the frequency domain analysis. But, the theory of frequency domain can not completely solve the aliasing problem in pulse Doppler and color Doppler.

Comparing lower frequency ultrasound, higher frequency ultrasound has larger attenuation coefficient and thus is more readily absorbed in medium, which limits the depth of penetration. So, if there is way to increase the penetration of higher frequency ultrasound?

### BRIEF SUMMARY OF THE INVENTION

Emitted from activated PZT, ultrasound pulses contain their quantity and size. Unlike the changes of sounds from coming or leaving motorcycle, the distance of motorcycles is changing. But more like playing table tennis, the racket hits the ball at fix location and changes the speeds of reflected balls, which changes the time of flight of the ball. No matter in continuous wave or pulse wave or color ultrasound, when checking the velocity of blood flow, the ultrasound system always detect the reflected ultrasound pulses from the fixed locations where the ultrasound beams cross with blood vessels to calculate the shift of time of flight (TOF) of ultrasound pulses. So, the detecting the velocity of moving objects can be calculated based on the TOF shift.

One aspect of the invention, correcting the transmitting distance of ultrasound pulses can rectify the registration of detecting depth, which improves the quality of ultrasound images. During the transmission in the medium, the speed of ultrasound pulse will gradually reduce due to the loss of its energy caused by acoustic impedance. The current calculation of ultrasound reflecting depth is based on fixed ultrasound speed, which will cause miscalculation of reflecting depth due to the change of actual ultrasound speed, and distort the ultrasound images. Because the ultrasound speed is inversely correlated to acoustic impedance and attenuation coefficient, the ultrasound speed reducing coefficient could be used to correct the registration of detecting depth, which will improve the quality of images.

In another aspect of the invention, the thickness of activated piezoelectric crystals (PZT) decides the quantity of ultrasound pulses, which affects their penetrating depth. The penetrating depth of ultrasound pulses is not directly related to their frequencies, but related to their quantity. The thicker PZT is, the greater quantity the ultrasound pulses will be and deeper the penetrating depth will be. Because the frequency is directly proportional to the sound speed in PZT elements, selecting PZT elements with higher speed in the PZT elements will generate the ultrasound pulses with more density and higher frequency and greater quantity. So, the pulses with higher frequency and greater quantity have deeper penetrating ability, which will improve the quality of images for high frequency ultrasound.

Another aspect of the invention is detecting the velocity of moving objects by TOF shift of time domain analysis for continuous wave, pulse wave and color ultrasound. It is based on the speed changes of reflected ultrasound pulses by the moving objects, which change their TOF and TOF shift. The moving objects may change the rebounding forces to the ultrasound pulses. The velocity and angle of moving objects with ultrasound beams decide rebounding force. Faster forward velocity and/or greater angle of moving object with ultrasound beam will generate greater rebound force, which create greater reflecting speed of ultrasound pulses and result in shorter TOF and greater TOF shift. On the contrary, faster reversed velocity and/or smaller angle of moving object with ultrasound beam will reduce rebound force, which produce slower reflecting speed of ultrasound pulses and result in longer TOF and greater TOF shift. For continuous, pulse wave and color Doppler, The velocity of moving objects can be calculated based on the related TOF shift.

Another aspect of the invention is clarifying the mechanism of aliasing and the ways of completely correcting the aliasing for pulsed wave and color ultrasound. If the speed of moving object is too fast, which makes TOF excesses its aliasing limit, the ultrasound system will misinterpret the reflected ultrasound pulse and generate aliasing TOF. For forward moving object, the aliasing limit for TOF is less than the value of half of calculated TOF. If TOF is smaller than the aliasing limit, the ultrasound system will misinterpret the reflected ultrasound pulse and add a value of calculated TOF into actual TOF, which generates aliasing TOF. Then the aliasing TOF is greater than calculated TOF. So, aliasing TOF shift is below baseline, which represent the moving object toward reversed direction. For reversely moving object, its TOF aliasing limit is greater than the value of one and half calculated TOF. If the actual TOF is greater than the aliasing limit, the ultrasound system will subtract a value of calculated TOF from actual TOF. Then the aliasing TOF is smaller than calculated TOF. So, aliasing TOF shift is above the baseline, which represent the moving object as forward direction. A computer program is used to identify the aliasing TOF shift, and the corrected TOF shift can be also used to correct aliasing when TOF excesses its aliasing limit in pulse wave or color ultrasound no matter how fast the velocity of moving objects will be. Correcting aliasing also avoid the interference of aliasing to the disturbed flow.

Based on the speed reduction of ultrasound pulses during the transmission, the quantity of ultrasound pulses, and the TOF changes of ultrasound pulses by the velocity of moving objects, TOF shift can more accurately present the relationship between the TOF of ultrasound pulses and the velocity of moving objects, and it also better explains the effect of the velocity and angle of the moving objects on the ultrasound pulses. So, the calculated velocity of moving objects from TOF shift should be more accurate than the results from Doppler shift.

### BRIEF DESCRIPTION OF THE SEVERAL VIEWS OF THE DRAWING

FIG. 1 is a schematic illustration of rebound force of forward flow to ultrasound pulse

FIG. 2 is a schematic illustration of rebound force of reversed flow to ultrasound pulse

FIG. 3 is a schematic illustration of spectrum for TOF shift of continuous wave ultrasound

FIG. 4 is a schematic illustration of aliasing TOF and TOF shift

FIG. 5a is a schematic illustration of aliasing TOF shift for forward flow of pulsed wave ultrasound

FIG. 5b is a schematic illustration of corrected TOF shift for forward flow of pulsed wave ultrasound

FIG. 6a is a schematic illustration of aliasing TOF shift for reversed flow of pulsed wave ultrasound

FIG. 6b is a schematic illustration of corrected TOF shift for reversed flow of pulsed wave ultrasound

FIG. 7 is a schematic illustration of computer program to calculate TOF shift of continuous wave ultrasound

FIG. 8 is a schematic illustration of computer program to identify and correct aliasing, and calculate TOF shift of pulsed wave or color ultrasound

### DETAILED DESCRIPTION OF THE INVENTION

The Speed of Ultrasound Pulses Gradually Reduces During Transmission

The transmission of ultrasound energy in the medium is more like pulses, which contain their quantity and size. Nowadays, the ultrasound speed is considered as fixed in the same medium. But, actually the speed of ultrasound pulses is not fixed at the same speed as supposed during the transmission, and it will gradually reduce due to the acoustic impedance of transmitting medium. As the bullets shooting from machine gun, their speed is gradually reduced due to the loss of their energy caused by the resistance of air. But, the frequencies of bullets at any sites of trajectory keep the same. The transmission of ultrasound pulses has the similar mechanism. During the transmission, the speed of ultrasound pulses gradually reduces due to the loss of their energy caused by the acoustic impedance, which will finally exhausts the energy of the ultrasound pulses. The rate of its speed change is decided by attenuation coefficient and acoustic impedance. But, Ultrasound pulses keep the same frequency during the transmission, including reflected frequency.

Currently, the attenuation coefficient is directly proportional to the ultrasound frequency. The lower the frequency is, the smaller the attenuation coefficient will be.

Attenuation Coefficient (dB/cm)=frequency (MHz)/2

Total attenuation (dB)=attenuation coefficient×distance

As the frequency is inversely proportional to the PZT thickness, the more PZT thickness has lower frequency.

frequency=sound speed in PZT/2×PZT thickness

In lower frequency ultrasound, more PZT materials will be activated from more thickness of PZT, it means that higher quantitative ultrasound pulses are generated from these more activated PZT materials.

So, attenuation coefficient is actually decided by the quantity of ultrasound pulses, which is related the thickness of activated PZT. Like heavier ball with more ability of traveling distance, the ultrasound pulses with higher quantity will have lower attenuation coefficient, and have smaller reducing rate of its speed comparing to the ultrasound pulses with smaller quantity.

The acoustic impedance is decided by the density of medium and ultrasound speed in the medium.

Acoustic impedance (rayls)=density (kg/m³)×speed (m/s)

So, higher the speed of ultrasound pulse is, the greater the acoustic impedance will be, which consume more its energy during the transmission. As the energy transmission of acoustic pulses in the medium, the speed of ultrasound pulses should be directly related to its energy, and its speed will gradually reduce due to the loss of its energy during its transmission until the exhaustion of its energy.

Rectifying the Registration of Detecting Depth of Ultrasound Pulses can Improve the Quality of Images

One aspect of the invention is rectifying the errors of the registration of detecting depth due to speed reduction of ultrasound pulses. Because ultrasound speed is affected by acoustic impedance and attenuation coefficient, its speed will gradually reduce as it transmits in the medium. Nowadays, the calculated detecting depth is based on fixed general ultrasound speed in the medium, which will cause miscalculation of detecting depth due to the change of actual ultrasound speed, and distort the ultrasound images. The longer the ultrasound pulses travel, the slower the pulse speed will be. So, actually traveling depth of ultrasound pulses is smaller than calculated detecting depth because of the speed reduction of ultrasound during the transmission. As the reduction of ultrasound speed is directly correlated to acoustic impedance and attenuation coefficient. The ultrasound speed reducing coefficient could be used to correct the errors of calculated detecting depth. The distance shift of ultrasound pulses is directly related of the speed reducing coefficient and traveling time of ultrasound. V is the general speed of ultrasound pulses in the transmitting medium. For instance, the general ultrasound speed in soft tissues is 1540 meter/second. t is the traveling time from emitting to receiving the ultrasound pulses. Calculated depth is the depth based on general ultrasound speed and traveling time of ultrasound pulses between emitting to receiving.

Speed reducing coefficient=acoustic impedance×attenuation coefficient

Depth shift=speed reducing coefficient×t/2

Calculated detecting depth=V×t/2

Corrected detecting depth=calculated depth−depth shift

The Quantity of Ultrasound Pulses Affect their Penetrating Depth

In another aspect of the invention, creating the transducer with more thickness of PZT and faster speed of sound in PZT will generate the ultrasound pulses with higher frequency and higher quantity, which increase their penetrating depth and improve the quality of images. The ultrasound transducers contain piezoelectric (PZT) materials, which emit ultrasound pulses. In the pulsed wave transducer, the PZT thickness and the speed of sound in PZT are related to the transducer frequency. The more thickness and/or slower speed of sound in PZT will induce lower frequency. Actually thicker activated PZT elements will emit greater quantity of the pulses comparing thinner PZT transducer, which explains why the ultrasound pulses with lower frequency have greater penetrating depth. At the same time, increasing the speed of sound in PZT also increases the density of the ultrasound pulses, which can increase the frequency of transducer.

frequency=sound speed in PZT/2×PZT thickness

Moving Objects Will Change TOF and Generate TOF Shift for Ultrasound Pulses

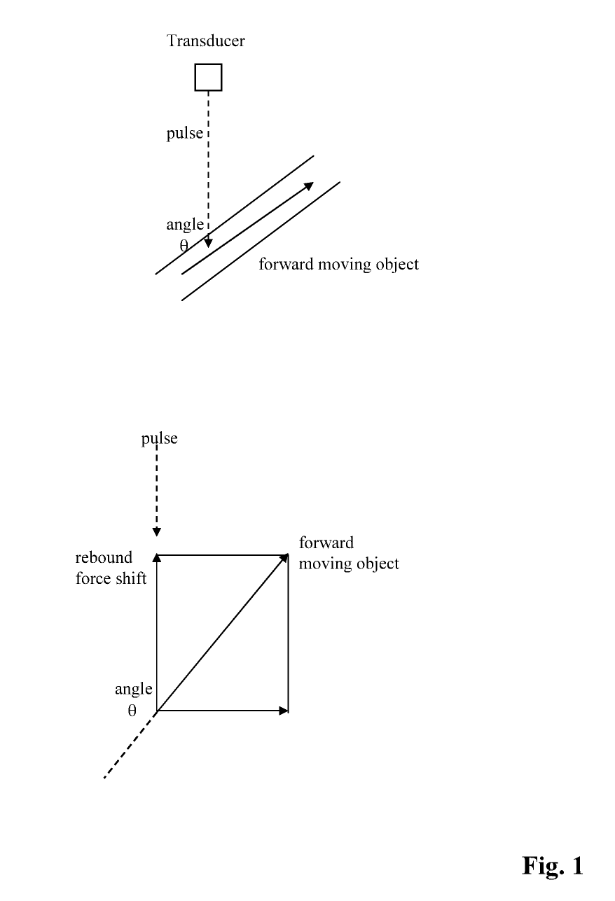

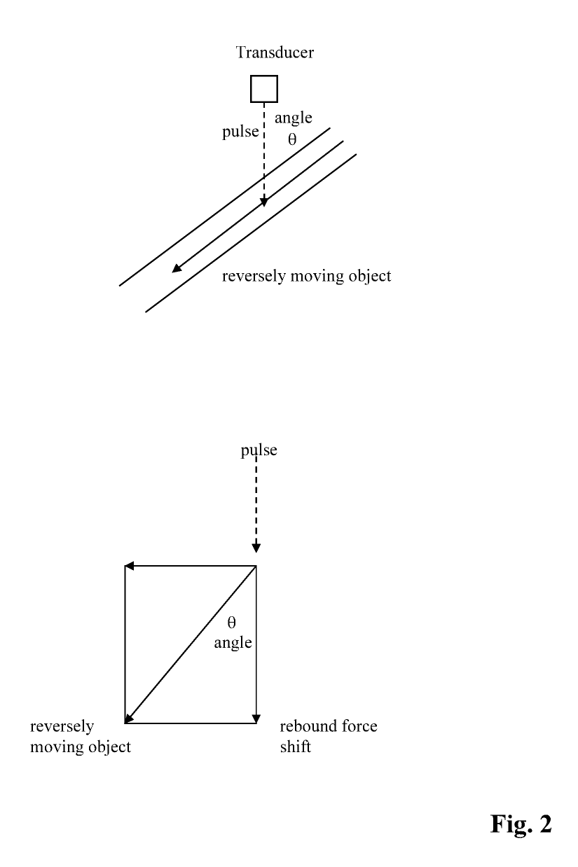

Ultrasound pulses can be reflected by motionless or moving objects. Comparing to motionless objects, moving objects will change the rebound force to the ultrasound pulses. The velocity and angle of moving objects with ultrasound beams will generate rebounding force shift. As in the FIG. 1, the forward moving objects will generate rebound force shift against ultrasound pulses. The rebounding force shift is decided by the speed and the angle θ of moving object with ultrasound beam. The faster speed of moving object and/or greater angle θ will generate greater forward rebounding force shift, which creates greater rebounding force and reflected speed of ultrasound pulses. As the result, its TOF is reduced and TOF shift is increased. On the contrary, as in the FIG. 2, reversely moving objects will generate rebounding force shift with the same direction of emitted ultrasound pulses, which reduces the rebounding force. The faster speed of moving objects and smaller angle θ will generate greater reversely rebounding force shift, which reduces rebounding force and reflected speed of ultrasound pulses. This will increase its TOF and TOF shift.

The quantity of ultrasound pulses also affects their TOF and TOF shift. As the quantity of ultrasound pulses is directly related the PZT thickness, it is inversely related to the TOF shift. The ultrasound pulses with greater quantity have smaller speed changes of reflected pulses, which elongates its TOF and decreases TOF shift. On the contrary, smaller quantitative pulses have the greater speed changes of reflected pulses, which shortens its TOF and increases TOF shift.

The sound speed in PZT also affects the TOF shift. The higher speed of sound speed in PZT generates higher density of the pulses, which will shorten its TOF and increase its TOF shift.

$$transducer frequency = sound speed in PZT / 2 \times PZT thickness$$

$$Rebounding force shift = speed of objects \times cos θ$$

$$Reflected speed shift = rebounding force shift / PZT thickness$$

$$TOF shift = \frac{2 \times speed of objects \times transducer frequency \times cos θ}{pulse propagation speed}$$

$$TOF shift = \frac{speed of objects \times cos θ \times sound speed in PZT}{PZT thickness \times pulse propagation speed}$$

$$TOF shift = \frac{Rebounding force shift \times sound speed in PZT}{PZT thickness \times pulse propagation speed}$$

$$TOF shift = \frac{reflected speed shift \times sound speed in PZT}{pulse propagation speed}$$

So, one aspect of the invention is calculating the velocity of moving objects based on TOF shift for continuous or pulsed wave ultrasound. As mentioned above, the speed of ultrasound pulses will gradually reduce, and moving objects will generate rebounding force shift, which changes the speed of reflected ultrasound pulses and their TOF. Therefore, TOF shift can more accurately present the relationship between the velocity of moving objects and the characters of ultrasound pulses.

Calculating the Velocity of Moving Objects by TOF Shift of Continuous Wave Ultrasound

Currently, it is considered that the speed of ultrasound is fixed in the same medium during the transmission. The moving objects will change the frequency of reflected ultrasound waves. As forward moving objects will compress the reflected frequency, which is higher than the emitted frequency. Its Doppler shift is above the baseline. So, calculating continuous wave (CW) Doppler shift is based on the difference between reflected frequency and emitted frequency.

Doppler shift=reflected frequency−emitted frequency

Doppler shift=2×speed of objects×transducer frequency×cos θ/propagation speed

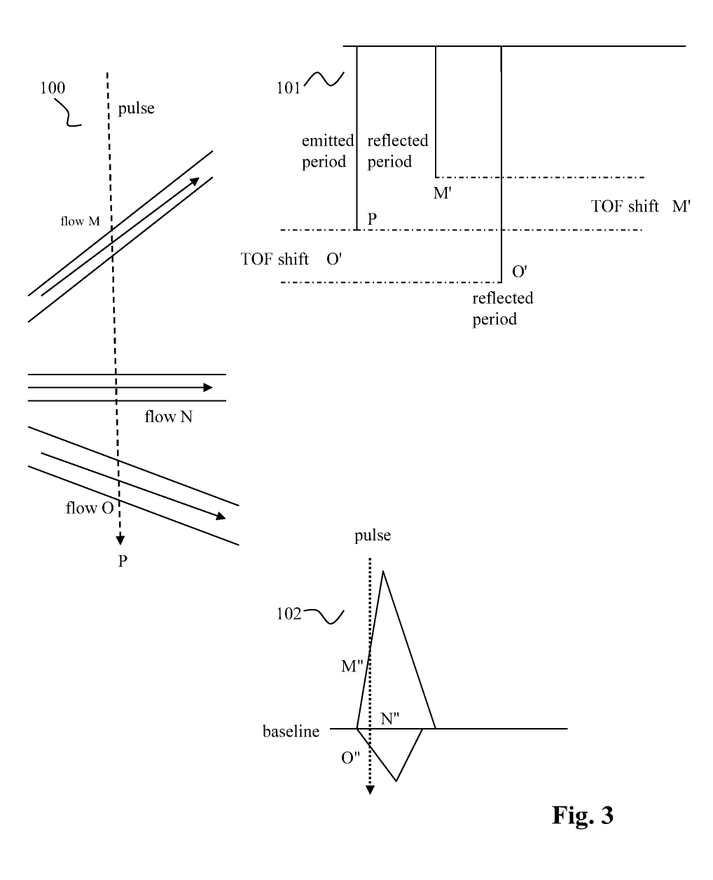

The present invention discloses that TOF shift in continuous wave ultrasound is the difference between the time of emitting period and the time of reflected period. As 101 in FIG. 3, the emitted period is the time between previously and following emitted pulses. The period is inversely proportional to its frequency. The reflected period is the time between previous and following reflected ultrasound pulses. if the ultrasound pulses are reflected from moving objects that is vertical to the ultrasound beam (flow N), the emitted period equals to its reflected period. But, if the ultrasound pulses are reflected from forward moving objects (flow M), their reflected pulse speeds will be accelerated due to increasing rebounding forces, which shorten their TOF M′. So, the reflected periods will be less than the time of emitted period, which generates TOF shift M′. On the contrary, for the reversely moving objects (flow O), its TOF O′ will be elongated due to reduced rebounding force and reflected ultrasound speed, and the reflected periods will be greater than the time of emitted period, which generates TOF shift O. The TOF shift equals to the time of emitted period subtracting the time reflected period.

emitted period=1/emitted frequency

reflected period=1/reflected frequency

reflected period=the time between previous and following reflected pulses

TOF shift=emitted period−reflected period

TOF shift=2×speed of objects×transducer frequency×cos θ/propagation speed

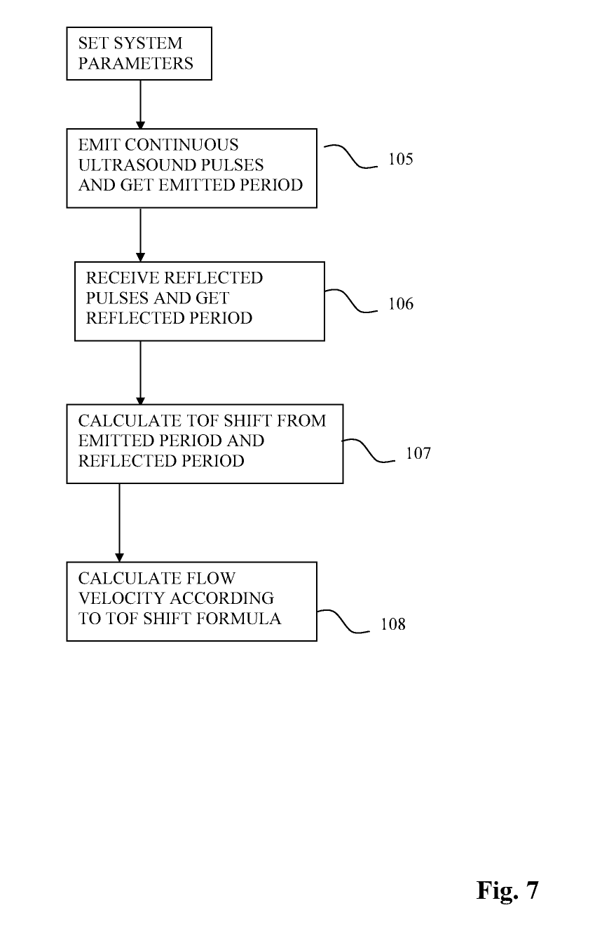

There are two PZT parts in CW ultrasound transducer. As in the FIG. 7, one PZT part emits the continuous ultrasound pulses with certain emitted period (105), which is decided by the ultrasound system. Another PZT part receives reflected ultrasound pulses and detects the time of reflected period between previous and following reflected ultrasound pulses (106). The time of reflected period is decided by the velocity of moving objects. Then, the ultrasound system gets TOF shift from the difference between emitted period and reflected period, and calculates the velocity of the moving objects according to the formula of TOF shift (108).

As 100 in FIG. 3, for CW ultrasound, the transducer receives all of the reflected ultrasound pulses from the area under the transducer. If there are several objects with different velocities toward transducer, they will rebound the ultrasound pulses with different reflected speeds and TOF, which generate different TOF shifts related to these moving objects. Then ultrasound system will compare the list of these reflected pulses and respectively present these TOF shifts on TOF shift spectrum as 102 in FIG. 3. Because for CW ultrasound, there are usually multiple moving objects under transducer with different velocities, such as multiple blood vessels, its TOF shift spectrum often presents as spectral broadening.

Calculating the Velocity of Moving Objects by TOF Shift of Pulse Wave and Color Ultrasound

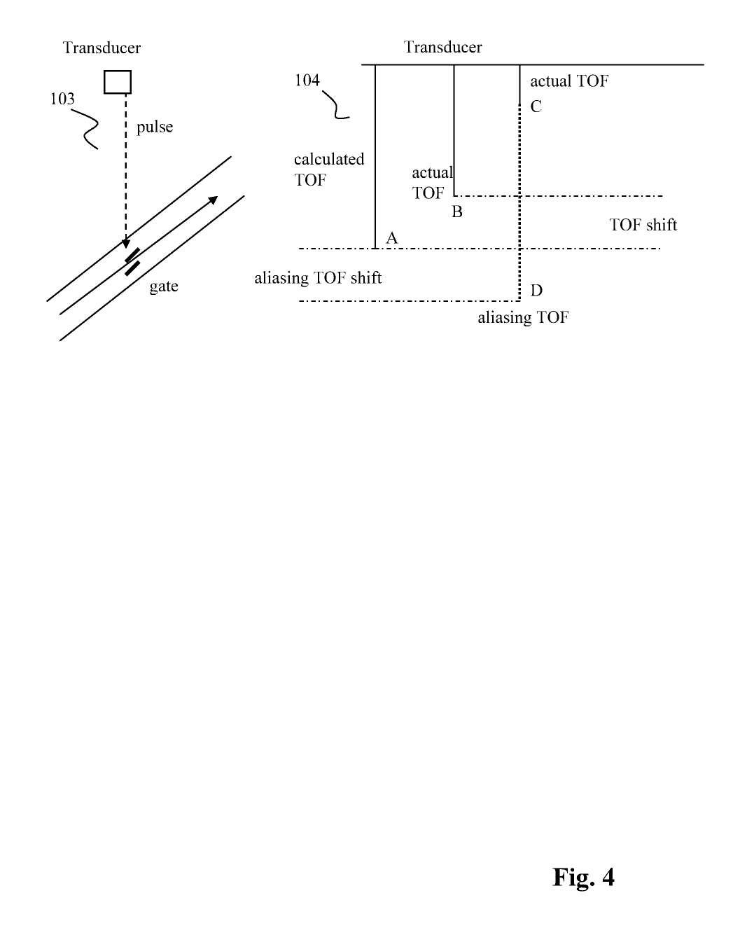

there is only one PZT part in pulsed wave ultrasound transducer, which sends and receives ultrasound pulses. So, the transducer has to receive previous reflected pulses before sending following emitted pulses. In order to detect the velocity of moving objects, the gate or detecting depth must be set as 103 in FIG. 4. So, based on the general speed of ultrasound pulses and the distance between the transducer and the gate, the calculated TOF can be obtained as A in FIG. 4. The actual TOF is the time the ultrasound pulses actually travel between the transducer and the gate, which can be affected by moving objects as B in FIG. 4. The TOF shift is the difference between calculated TOF and actual TOF. For forward moving object, it accelerates the speed of reflected pulses, which shorten actual TOF. So, calculated TOF is greater than actual TOF, the TOF shift is positive, which is above baseline. On the contrary, reversely moving objects elongate actual TOF, and TOF shift is negative, which is below the baseline.

TOF shift=calculated TOF−actual TOF

TOF shift=2×speed of objects×transducer frequency×cos θ/propagation speed

The actual TOF is usually the detected time between emitting and receiving pulses by ultrasound system. But if the velocity of moving objects is too fast, and makes its TOF excesses its aliasing limit, the ultrasound system will misinterpret it and produce aliasing TOF and aliasing TOF shift, which present the moving objects as toward opposite direction.

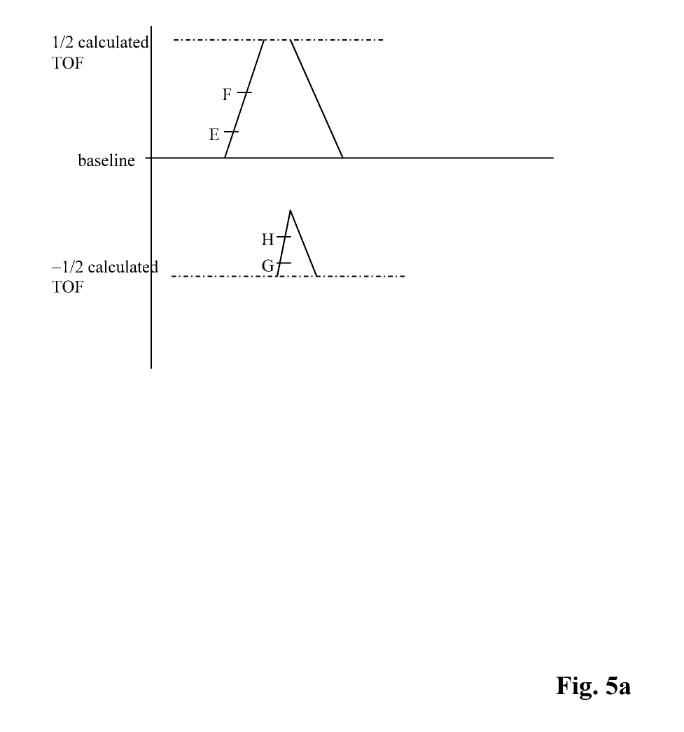

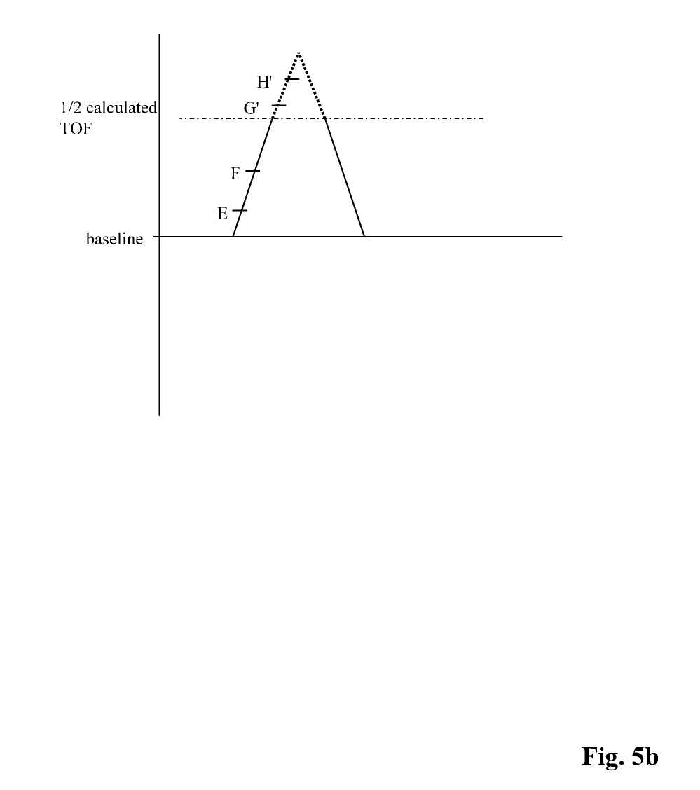

For the forward moving object, its aliasing limit of TOF is less than half value of calculated TOF. if the actual TOF is less than half of calculated TOF, the ultrasound system will misinterpret the reflected pulse, and add a calculated TOF value in aliasing TOF, which is larger than calculated TOF as 104 in FIG. 4. So, the aliasing TOF shift becomes negative, which misrepresents the objects moving toward reversed direction. As the result, it discontinues the profile of TOF shift. Before TOF excesses its aliasing limits, the value of TOF shift is above the baseline (from E to F in FIG. 5). But, after actual TOF excesses its aliasing limit, the value of aliasing TOF shift is below the baseline (from G to H in FIG. 5).

Aliasing TOF=actual TOF+calculated TOF

Aliasing TOF shift=calculated TOF−aliasing TOF

Aliasing TOF shift=−actual TOF

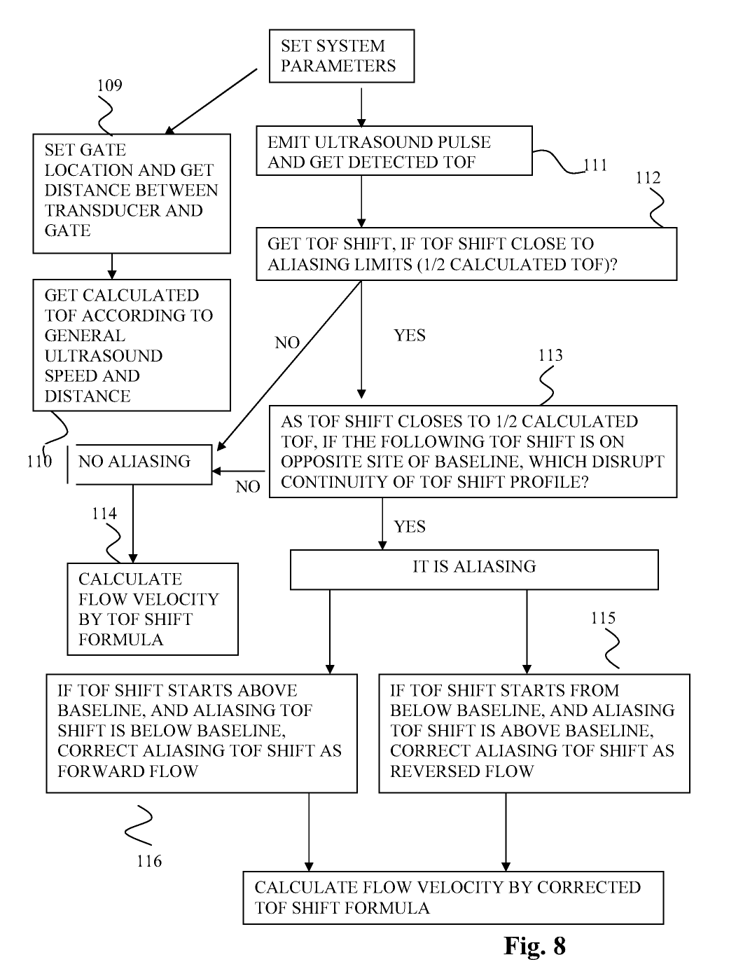

So, in the invention, a computer program is designed to identify and correct the aliasing. As the value of TOF shift approaches the half value of calculated TOF, the computer program will compare the value o following TOF shift with the value of previous TOF shift. If the value of following TOF shift is located on opposite site of baseline to the value of previous TOF shift. There is a TOF shift. After identifying the aliasing TOF shift, the ultrasound system will rectify the registering corrected TOF shift by subtracting the value of aliasing TOF shift from calculated TOF (116 in FIG. 8).

TOF shift=calculated TOF−actual TOF

Aliasing TOF shift=−actual TOF

corrected TOF shift=calculated TOF−|aliasing TOF shift|

After rectifying the registering errors of TOF shift, the value of corrected TOF shift will increase as the value of TOF decreases, which reestablish the continuation of profile of TOF shift (FIG. 5B), and the value of correct TOF shift can be used to calculated the velocity of moving objects.

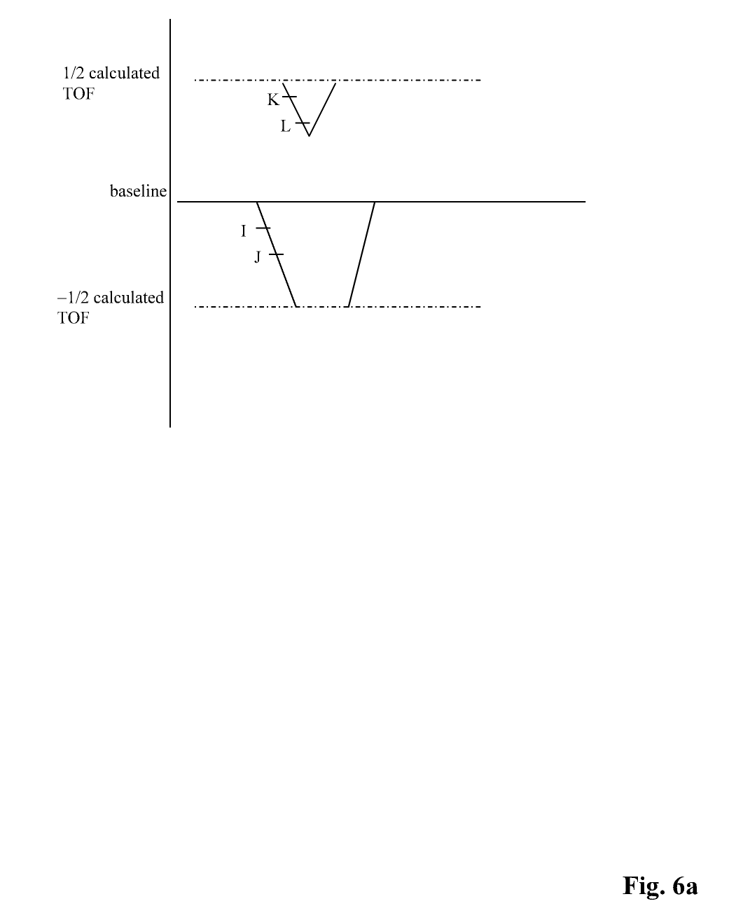

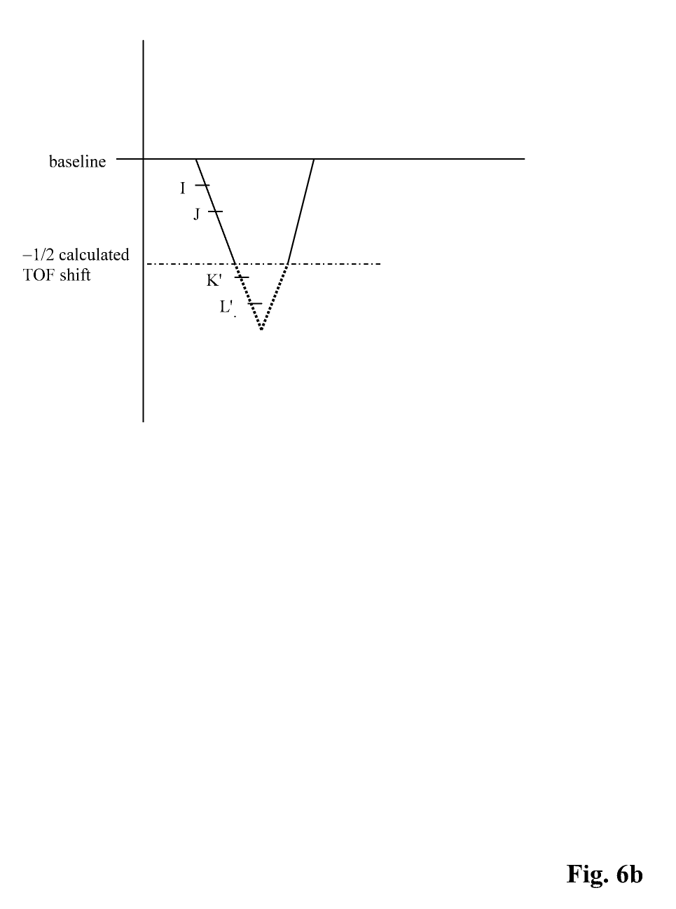

For the reversely moving objects, the rebounding force is reduced, which decreases the reflected speed of ultrasound pulses and increases their TOF, which is greater than the value of calculated TOF. So, the value of TOF shift is below the baseline. The aliasing limit of TOF for reversely moving objects is the value of actual TOF larger than the value of one and half calculated TOF. If the value of actual TOF excesses its aliasing limit, the ultrasound system will misinterpret the reflected ultrasound pulses and subtract a calculated TOF value in aliasing TOF, which is smaller than calculated TOF. So, aliasing TOF shift will be above baseline, which misrepresents the moving objects from forward direction. As a result, the continuity of the profile of TOF shift is disrupted (FIG. 6a). In the invention, a computer program is designed to identify the aliasing. As the value of TOF shift approaches the value of half of calculated TOF, and following TOF shift is on the opposite site of baseline and above baseline, the following TOF shift is the aliasing TOF shift.

Aliasing TOF=actual TOF−calculated TOF

Aliasing TOF shift=calculated TOF−aliasing TOF

Aliasing TOF shift=2×calculated TOF−actual TOF

After identifying the aliasing TOF shift, the computer program will rectify the aliasing TOF shift by subtract the value of a calculated TOF from the value of aliasing TOF shift, which is based on following equations:

TOF shift=calculated TOF−actual TOF and

aliasing TOF shift=2×calculated TOF−actual TOF

correct TOF shift=aliasing TOF shift−calculated TOF

After correcting aliasing TOF shift, their correct registration will be established, which continues the profile of its TOF shift (FIG. 6b).

Increasing the Quantity of Ultrasound Pulses or Reducing Detecting Depth Helps Improving Aliasing

Increasing the quantity of ultrasound pulses will decrease the speed changes of reflected pulses, which increase their TOF and delays their value reaching their aliasing limits.

1. Rectifying the errors of ultrasound traveling distance caused by its speed reduction registers correct detecting depth. During the transmission, the ultrasound speeds gradually reduce due to the loss of their energy caused by the acoustic impedance. So, calculation of detecting depth according to fixed ultrasound speed will distort images. Correctly registering the errors of detecting depth due to the speed reduction of ultrasound pulses during the transmission improves the quality of images.

2. The method of claim 1, wherein ultrasound speed reducing coefficient can be used to correct the registration of the detecting depth. The Ultrasound speed reducing coefficient equals to the product of acoustic impedance and attenuation coefficient. The depth shift of ultrasound pulses equals to the half value of the product of speed reducing coefficient and traveling time. V is the general speed of ultrasound pulses in the transmitting medium. t is the traveling time between transmitting and receiving pulses. Calculated depth is the depth based on general ultrasound speed and traveling time of ultrasound pulses between transmitting and receiving.
Speed reducing coefficient=acoustic impedance×attenuation coefficient
Depth shift=speed reducing coefficient×t/2
Calculated depth=V×t/2
Corrected detecting depth=calculated depth−depth shift

3. The thickness of activated piezoelectric materials is directly related to the length and quantity of ultrasound pulses, and increasing the thickness of activated piezoelectric materials will generate the longer ultrasound pulses with greater quantity.

4. The method of claim 3, wherein increasing the quantity of ultrasound pulses increase their penetrating depth.

5. The method of claim 4, wherein the ultrasound speed in PZT elements affects the length and density of ultrasound pulses, and selecting thicker PZT elements with faster sound speed in the PZT elements will generate ultrasound pulses with higher frequency and greater quantity, which increase their penetrating depth.

6. The method of claim 3, wherein adjusting the quantity of ultrasound pulses affects the changes of their reflected speed. The quantity of ultrasound pulses is inversely related to the changes of their reflected speed. For the forward moving objects, the ultrasound pulses with greater quantity have the smaller increased speed of reflected pulses. On the contrary, smaller quantity ultrasound pulses have greater increased speed of reflected pulses. For the reversely moving objects, increasing pulse quantity will have less decreased speed of reflected pulses, and reducing the pulse quantity will have more decreased speed of reflected pulses. The changes of reflected speed of ultrasound pulses will affect their TOF and TOF shift.
$$T O F shif t = \frac{speed of moving objects \times cos θ \times sound speed in PZT}{PZT thickness \times pulse propagation speed}$$

7. Using the shift of time of flight (TOF) of ultrasound pulses detects the velocity of moving objects. The moving objects can change the rebounding force to the ultrasound pulses. The rebounding force shift is decided by the velocity and/or angle of moving objects with ultrasound beam. The speed shift of reflected ultrasound pulses are decided by the rebounding force shift and the quantity of ultrasound pulses. The changes of reflected speed of ultrasound pulses will alter their TOF and TOF shift. The value of TOF shift can be used to calculate the speed of moving objects.
$$Rebounding force shift = speed of objects \times cos θ$$
$$Reflected speed shift = rebounding force shift \text{/} PZT thickness$$
$$T O F shif t = \frac{reflected speed shift \times sound speed in PZT}{pulse propagation speed}$$
$$T O F shif t = \frac{2 \times speed of moving object \times transducer frequency \times cosθ}{propagation speed}$$

8. The method of claim 7, wherein adjusting detecting angles changes the rebounding forces and TOF shift. For forward moving objects, faster velocity and/or smaller angle of moving objects with ultrasound beam will generate greater rebound force, which generate greater speed of reflected ultrasound pulses and result in shorter TOF. On the contrary, for reversely moving objects, faster velocity and/or smaller angle of moving object with ultrasound beam will reduce rebounding force, which generate slower speed of reflected ultrasound pulses and result in longer TOF. So, TOF shift value can be adjusted by changing the angles of moving objects with the ultrasound beams.

9. The method of claim 7, wherein adjusting the quantity of ultrasound pulses changes the TOF shift. The quantity of ultrasound pulses is directly proportional to the PZT thickness. Increasing the quantity of the ultrasound pulses will decrease the changes of reflected speed and elongate TOF, which reduces TOF shift. On the contrary, decreasing the quantity of the ultrasound pulses will increase the changes of reflected speed and shorten TOF, which increase TOF shift.
$$T O F shif t = \frac{speed of objects \times cos θ \times sound speed in PZT}{PZT thickness \times pulse propagation speed}$$

10. The method of claim 7, wherein selecting the PZT elements with different sound speed in the PZT elements affects TOF shift. Higher speed in the selected PZT elements will generate the greater TOF shift.
$$T O F shif t = \frac{speed of objects \times cos θ \times sound speed in PZT}{PZT thickness \times pulse propagation speed}$$

11. The method of claim 7, wherein for each reflected pulses of continuous wave Doppler, the TOF shift equals to the time of emitted period subtracting the time of reflected period. The time of reflected period equals the time between previous and following reflected pulses.
TOF shift=emitted period−reflected period
TOF shift=2×speed of objects×transducer frequency×cos θ/propagation speed

12. The method of claim 7, wherein for each reflected pulses of pulse wave or color Doppler, the TOF shift is the difference between calculated TOF and actual TOF. The calculated TOF is the calculated time that ultrasound pulse travels between the transducer and gate according to the distance and general ultrasound speed in the transmitting medium. The actual TOF is the actual time that the ultrasound pulses travel between transducer and the gate.
TOF shift=calculated TOF−actual TOF
TOF shift=2×speed of objects×transducer frequency×cos θ/propagation speed

13. The method of claim 11, for correcting aliasing TOF shift of pulse wave or color Doppler, the method comprising:
identifying an aliasing of a pulse wave or color Doppler, wherein a TOF shift of the pulse wave or color Doppler is determined based on TOF of reflected ultrasound pulse; and
rectifying a misinterpreted TOF of reflected ultrasound pulse to correct a location of the reflected ultrasound pulse by registering an aliasing TOF shift after an actual TOF exceeds the aliasing limit, no matter how fast the velocity of the objects will be.

14. The method of claim 13, wherein for a forward flow, if the actual TOF is less than half of calculated TOF, the reflected ultrasound pulse is misinterpreted as from a previous emitted ultrasound pulse, and the pulse wave Doppler comprises a computer program to add one calculated TOF to the actual TOF, and wherein an aliasing TOF is greater than the calculated TOF:
aliasing TOF=calculated TOF+actual TOF and
aliasing TOF shift=calculated TOF−aliasing TOF=−actual TOF

15. The method of claim 13, wherein the correctly registering of the aliasing TOF shift for the forward object comprises:
determining a corrected TOF shift for the forward object by subtracting a value of the aliasing TOF shift from the calculated TOF, based on an equation:
TOF shift=calculated TOF−actual TOF,
aliasing TOF shift=−actual TOF, wherein the value of the aliasing TOF shift equals to a value of the actual TOF
corrected TOF shift=calculated TOF−|aliasing TOF shift|

16. The method of claim 13, wherein for a reversely moving object, if the actual TOF is greater than one and half of calculated TOF, the reflected ultrasound pulse is misinterpreted as from a following emitted ultrasound pulse, and the pulse wave Doppler comprises a computer program to subtract one calculated TOF to the actual TOF, and wherein an aliasing TOF is smaller than the calculated TOF:
$$aliasing T O F = actual T O F - calculated T O F and$$
$$aliasing T O F shift = calculated T O F - aliasing T O F = \\ 2 calculated - actual T O F$$

17. The method of claim 13, wherein the correctly registering of aliasing TOF shift for the reversely moving objects comprises:
determining a corrected TOF shift by subtracting the calculated TOF from a value of the aliasing TOF shift for the reversely moving objects, based on equations:
TOF shift=calculated TOF−actual TOF and
aliasing TOF shift=2 calculated TOF−actual TOF
So, after TOF of reversely moving objects exceeds one and half calculated TOF, the registering of the aliasing TOF shift is:
corrected TOF shift=aliasing TOF shift−calculated TOF

18. The method of claim 6, wherein increasing the quality of ultrasound pulses increases their TOF and delays their value reaching their aliasing limits, which will help to correct aliasing.

19. The method of claim 7, wherein a method for calculating the velocity of a moving object is based on TOF shift comprising:
Calculating the velocity of a moving object using a TOF shift equation, wherein 0 is an angle of a ultrasound beam made with a moving object, the propagation speed is determined by a medium for ultrasound pulses, and the TOF shift is the determined TOF shift for continuous wave, pulse or color Doppler:
$$T O F shif t = \frac{2 \times speed of moving object \times transducer frequency \times cosθ}{propagation speed}$$

20. The method of 19, wherein for calculating the velocity of a moving object after the actual TOF exceeds the aliasing limit of the pulse wave or color ultrasound, the TOF shift is the corrected TOF shift based on the aliasing TOF shift provided by the pulse wave or color Doppler.
